(19) United States
(12) Patent Application Publication
(10) Pub. No.: US 2023/0405154 A1
(43) Pub. Date: Dec. 21, 2023

(54) METHOD FOR PREPARING NANOPARTICLES

(71) Applicants: NH THERAGUIX, Meylan (FR); UNIVERSITE CLAUDE BERNARD LYON 1, Villeurbanne (FR); CENTRE NATIONAL DE LA RECHERCHE SCIENTIFIQUE - CNRS -, Paris (FR)

(72) Inventors: Olivier TILLEMENT, Fontaines Saint-Martin (FR); François LUX, Lyon (FR); Fabien ROSSETTI, Villeurbanne (FR); Paul ROCCHI, Lyon (FR); Tristan DOUSSINEAU, Lyon (FR)

(21) Appl. No.: 18/253,586

(22) PCT Filed: Nov. 19, 2021
(86) PCT No.: PCT/FR2021/052041

§ 371 Date: May 18, 2023

(30) Foreign Application Priority Data
Nov. 19, 2020 (FR) 2011904

(51) Int. Cl. A61K 49/18; A61K 51/12

(52) U.S. Cl. CPC A61K 49/1881; A61K 51/1244; B82Y 5/00

(57) ABSTRACT

The present disclosure relates to nanoparticles and the uses thereof in medicine, in particular for the treatment of tumours.

### TECHNICAL FIELD

The present disclosure relates to a process for preparing nanoparticles, to nanoparticles and the uses thereof in the field of medicine, in particular for the treatment of tumors.

### PRIOR ART

The fight against cancer is currently based on three main therapeutic methods: surgery, chemotherapy and radiotherapy. Increasingly, research is currently focusing on the use of nanoparticles due to their abilities to improve these 3 types of treatment, but also to offer an improvement in imaging techniques or even to combine imaging and therapy leading to the concept of theranostics. For radiotherapy, it is thus possible to increase the effect of the dose locally while sparing the surrounding healthy tissues owing to the presence of elements with a high atomic number.

Despite their advantageous multimodal properties and strong preclinical research, only a small number of nanoparticles have currently reached the clinical phase (Lux et al., 2018, Br. J. Radiology, 2018, 91, 20180365).

This number is even lower for the nanoparticles injected intravenously for which mention may be made of AuroShell having a silica core and a gold shell of around 155 nm, CUT-6091 consisting of a pegylated gold core for drug delivery, NU-0129 consisting of gold nanoparticles for nucleic acid delivery, Cornell Dots consisting of fluorescent polysiloxane for melanoma targeting, and AGuIX consisting of nanoparticles based on polysiloxane and gadolinium chelates for radiotherapy and MRI imaging.

Among these nanoparticles, two of them (AGuIX and Cornell Dots) have a hydrodynamic diameter of less than 10 nm allowing the renal excretion thereof after intravenous administration. Ultrafine nanoparticles are particularly suitable for clinical use owing to this rapid renal excretion that limits possible toxicity but also due to better tumor penetration and, in the case of radiosensitization, to “nanoscale dose deposition” effects that locally increase the efficacy of the delivered dose.

The presence of chelates on the surface of a nanoparticle is often necessary for the chelation of metal ions useful in imaging or in therapy. This is the case for the radioactive metal ions used in brachytherapy or in scintigraphy or for the magnetic ions used in MRI. Two main strategies currently exist in the literature for obtaining metal chelates or free chelates at the surface of the nanoparticle.

The first strategy for obtaining nanoparticles of this type is to carry out a synthesis by incorporating the chelates directly during the nanoparticle formation step. This is, for example, the strategy employed by N. G Chabloz et al. (Chem. Eur. J., 2020, 26, 4552-4566) to obtain gold nanoparticles functionalized by gadolinium complexes and by porphyrins for photodynamic therapy and MRI. This strategy can also be used for the one-pot synthesis of polysiloxane nanoparticles as proposed by V. L. Tran et al. 2018 (Mt. Chem. B, 2018, 6, 4821-4834).

These nanoparticles then have free chelates or chelates comprising gadolinium depending on the starting silanes used. However, this methodology has several shortcomings, the ratios and addition times must be determined with very great precision in order to have reproducible nanoparticles, which makes attempts at scale-up for clinical use tricky.

The second strategy consists in obtaining the desired nanoparticle then, via a post-functionalization step, in adding free chelates or chelates already comprising a metal. This is the strategy used by P. Bouziotis et al. (Nanomedicine, 2017, 12, 1561-1574) on AGuIX polysiloxane-based nanoparticles. NODAGA anhydride was functionalized on the surface of the nanoparticles by a reaction between the anhydride and the surface amines. Then galium 68 was added to be able to perform preclinical PET imaging. M. Pretze et al. (Journal of Labelled Compounds and Radiopharmaceuticals, 2019, 62, 471-482) used the same type of strategy on gold nanoparticles which were synthesized before the introduction of a maleimide function by ligand exchange reaction then the addition of NODAGA. The radioactive labeling with copper 64 took place in a final step. The main drawback of this strategy is that it greatly modifies the size of the nanoparticles and their surface in terms of surface charge and also of hydrophilicity/lypophilicity which can lead to a different biodistribution of the starting nanoparticle.

It therefore seems necessary to develop a process for generating free chelates on the surface of the nanoparticle which modifies the characteristics of the starting nanoparticle as little as possible, especially in the case of a nanoparticle already present in the clinic phase, such as the AGuIX nanoparticle, and having suitable biodistribution characteristics. Another objective of the present invention is thus to provide access to nanoparticles equivalent to a starting nanoparticle but a portion of the original chelates of which have been released, and which can then be left free or chelated with another metal of interest.

The present disclosure improves the situation with respect to one or more of these abovementioned objectives.

It is also proposed to use chelating nanoparticles having a suitable biodistribution in the treatment of tumors, in particular primary and/or metastatic tumors.

### SUMMARY

The invention relates to one of the embodiments described below or combinations thereof:

Embodiment 1: A process for preparing a colloidal solution of nanoparticles, each nanoparticle comprising chelating groups grafted onto a polymer matrix, one portion only of the chelating groups being complexed with a metal cation, the other portion being uncomplexed, said process comprising

(1) the synthesis or the provision of a colloidal solution of precursor nanoparticles, said precursor nanoparticles having the following formula [Ch-M₁]ₙ-PS wherein:
PS is an organic or inorganic polymer matrix, for example a polysiloxane matrix,
[Ch-M₁] is a chelating group complexed with a metal cation M₁ with a high atomic number Z greater than 40, and preferably greater than 50,
Ch is covalently grafted onto the surface of a polymer matrix, for example, a polysiloxane matrix,
n is between 5 and 100, and,
the average hydrodynamic diameter of the nanoparticles is between 1 and 50 nm, preferably between 2 and 20 nm, and more preferentially between 2 and 8 nm,
   (2) a step of treating the colloidal solution in an acid medium, for example by adding a hydrochloric acid solution, in order to obtain a pH preferably below 2.0, preferably below 1.0, for a time sufficient to obtain a partial release of the metal cations M₁,
   (3) where appropriate, a step of diluting the colloidal solution, for example with water,
   (4) a purification step to separate the nanoparticles obtained in step (2) from the metal cations M₁ released,
   (5) where appropriate, a step of concentrating the solution of the nanoparticles obtained in step (4),
   (6) where appropriate, repeating steps (3), (4) and (5),
   (7) where appropriate, freezing and/or freeze-drying the solution of nanoparticles obtained in one of steps (4), (5) or (6).

Embodiment 2: The process as claimed in embodiment 1, characterized in that M₁ is chosen from metal cations with a high atomic number Z greater than 40, and preferably greater than 50, in particular selected from radiosensitizers and/or contrast agents for magnetic resonance imaging (MRI), for example M₁ is chosen from gadolinium and bismuth.

Embodiment 3: The process as claimed in embodiment 1 or 2, characterized in that the chelating group Ch is chosen from macrocyclic agents, preferably from 1,4,7-triazacyclononane-triacetic acid (NOTA), 1,4,7,10-tetraazacyclododecane-1,4,7,10-tetraacetic acid (DOTA), 1,4,7-triazacyclononane-1-glutaric acid-4,7-diacetic acid (NODAGA), and 1,4,7,10-tetraazacyclododecane, 1-(glutaric acid)-4,7,10-triacetic acid (DOTAGA), 2,2′,2″,2′″-(1,4,7,10-tetraazacyclododecane-1,4,7,10-tetrayl)tetraacetamide (DOTAM), and 1,4,8,11-tetraazacyclotetradecane (Cyclam), 1,4,7,10-tetraazacyclododecane (Cyclen) and deferoxamine (DFO).

Embodiment 4: The process as claimed in any one of embodiments 1 to 3, characterized in that the chelating group Ch is DOTAGA of formula (I) below [Chem. 1]:

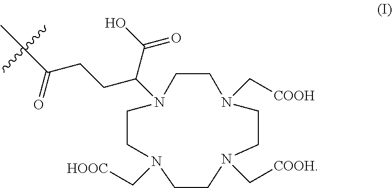 [SMILES: *C(=O)CCC(C(=O)O)N1CCN(CC(=O)O)CCN(CC(=O)O)CCN(CC(=O)O)CC1]

Embodiment 5: The process as claimed in one of embodiments 1 to 4, characterized in that PS is a polysiloxane matrix.

Embodiment 6: The process as claimed in embodiment 5, characterized in that the precursor nanoparticles have the following characteristics:
the weight ratio of silicon to the total weight of the nanoparticle is between 5% and 25%,
the total number n of chelating groups grafted onto the polymer is between 5 and 50 per nanoparticle, preferably between 10 and 30, and,
the nanoparticle has an average diameter of between 2 and 8 nm.

Embodiment 7: The process as claimed in any one of embodiments 1 to 6, characterized in that the precursor nanoparticles have the following characteristics:
(i) PS is a polysiloxane matrix,
(ii) Ch is a DOTAGA chelating group of formula (I) below [Chem. 1]

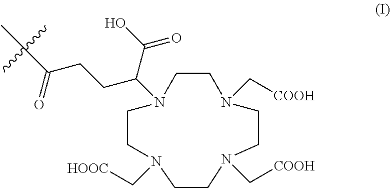 [SMILES: *C(=O)CCC(C(=O)O)N1CCN(CC(=O)O)CCN(CC(=O)O)CCN(CC(=O)O)CC1]

and grafted to the polysiloxane matrix by Si—C bond,
(iii) M₁ is the gadolinium cation Gd³⁺,
(iv) n is between 5 and 50, preferably between 10 and 30, and
(v) the average hydrodynamic diameter is between 2 and 8 nm.

Embodiment 8: A process for preparing a colloidal solution of nanoparticles, each nanoparticle comprising chelating groups grafted onto a polymer matrix, a first fraction f1 of the chelating groups being complexed with a metal cation M₁, a second fraction f2 being being complexed with a cation M₂, and a third fraction f3 being uncomplexed, said process comprising
(1) the synthesis or the provision of a colloidal solution of precursor nanoparticles, said precursor nanoparticles having the following formula [Ch-M₁]ₙ-PS wherein:
   PS is an organic or inorganic polymer matrix,
   Ch is a chelating group complexed with a metal cation M₁ with a high atomic number Z greater than 40, and preferably greater than 50,
   Ch is grafted onto the polymer matrix,
   n is between 5 and 100, and,
   the average hydrodynamic diameter of the nanoparticle is between 1 and 50 nm, preferably between 2 and 20 nm, and more preferentially between 2 and 8 nm
(2) a step of treating the colloidal solution in an acid medium, for example by adding a hydrochloric acid solution, in order to obtain a pH below 2.0, preferably below 1.0, for a time sufficient to obtain a partial release of the metal cations M₁,
(3) where appropriate, a step of diluting the solution, for example with water,
(4) a purification step to separate the nanoparticles obtained in step (2) from the free metal cations M₁,
(5) where appropriate, a step of concentrating the solution of the nanoparticles obtained in step (4),
(6) where appropriate, repeating steps (3), (4) and (5),
(7) optionally, a step of partial recomplexation of the nanoparticles obtained in step (2), (3), (4), (5) or (6) with a determined amount of metal cation M₁ in order to obtain a determined amount of chelating group Ch complexed with the metal cation M₁,
(8) bringing the solution of nanoparticles obtained in step (4), (5), (6) or (7) into contact with a sufficient amount of cation M2, for example a metal cation different from the metal cations M₁ or a radioisotope, to complex at least some of the chelating groups Ch1 freed in step (2) and,
(9) where appropriate, freezing and/or freeze-drying the solution of nanoparticles obtained in step (8).

Embodiment 9: The process as claimed in embodiment 8, characterized in that M₁ and/or M₂ are chosen from metal cations with a high atomic number Z greater than 40, and preferably greater than 50, in particular selected from radiosensitizers and/or contrast agents for magnetic resonance imaging (MRI), for example gadolinium or bismuth.

Embodiment 10: The process as claimed in embodiment 8 or 9, characterized in that the chelating group Ch is chosen from macrocyclic agents, preferably from 1,4,7-triazacyclononane-triacetic acid (NOTA), 1,4,7,10-tetraazacyclododecane-1,4,7,10-tetraacetic acid (DOTA), 1,4,7-triazacyclononane-1-glutaric acid-4,7-diacetic acid (NODAGA), and 1,4,7,10-tetraazacyclododecane, 1-(glutaric acid)-4,7,10-triacetic acid (DOTAGA), 2,2′,2″,2′″-(1,4,7,10-tetraazacyclododecane-1,4,7,10-tetrayl)tetraacetamide (DOTAM), and 1,4,8,11-tetraazacyclotetradecane (Cyclam), 1,4,7,10-tetraazacyclododecane (Cyclen) and deferoxamine (DFO).

Embodiment 11: The process as claimed in one of embodiments 8 to 10, characterized in that the chelating group Ch is DOTAGA of formula (I) below [Chem. 1]:

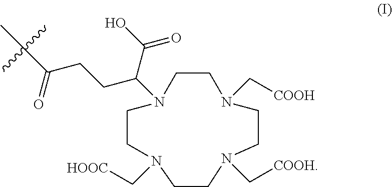 [SMILES: *C(=O)CCC(C(=O)O)N1CCN(CC(=O)O)CCN(CC(=O)O)CCN(CC(=O)O)CC1]

Embodiment 12: The process as claimed in one of embodiments 8 to 11, characterized in that PS is a polysiloxane matrix.

Embodiment 13: The process as claimed in embodiment 12, characterized in that the precursor nanoparticles have the following characteristics:
the weight ratio of silicon to the total weight of the nanoparticle is between 5% and 25%,
the total number n of chelating groups grafted onto the polymer is between 5 and 50 per nanoparticle, preferably between 10 and 30, and,
an average diameter of between 2 and 8 nm.

Embodiment 14: The process as claimed in any one of embodiments 8 to 13, characterized in that the precursor nanoparticles have the following characteristics:
(i) PS is a polysiloxane matrix,
(ii) Ch is a DOTAGA chelating group of formula (I) below [Chem.1]

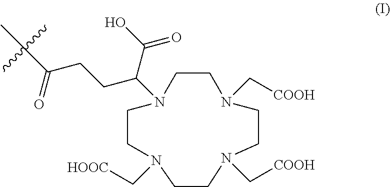 [SMILES: *C(=O)CCC(C(=O)O)N1CCN(CC(=O)O)CCN(CC(=O)O)CCN(CC(=O)O)CC1]

and grafted to the polysiloxane matrix by Si—C bond,
(iii) M₁ is the gadolinium cation Gd³⁺,
(iv) n is between 5 and 50, preferably between 10 and 30, and
(v) the average hydrodynamic diameter is between 2 and 8 nm.

Embodiment 15: The process as claimed in any one of embodiments 8 to 14, characterized in that the cation M₂ is chosen from scintigraphy imaging agents, for example ⁴⁴Sc, ⁶⁴Cu, ⁶⁸Ga, ⁸⁹Zr, ¹¹¹In, ^{99m}Tc.

Embodiment 16: The process as claimed in any one of embodiments 8 to 15, characterized in that the cation M₂ is chosen from therapeutic agents for brachytherapy, for example ⁹⁰Y, ¹⁶⁶Ho, ¹⁷⁷Lu, ²¹²Bi, ²¹³Bi, ²¹¹At.

Embodiment 17: The process as claimed in any one of embodiments 8 to 16, characterized in that f1 is between 0.1 and 0.9, f2 is between 0.1 and 0.9 and f3 is between 0 and 0.5, typically f1 is between 0.25 and 0.35, f2 is between 0.65 and 0.75, and f3 is substantially zero.

Embodiment 18: The process as claimed in any one of embodiments 8 to 17, characterized in that each nanoparticle is further functionalized with a targeting agent, in particular a peptide, an immunoglobulin, a nanobody, an antibody, an aptamer or a targeting protein.

Embodiment 19: A solution of nanoparticles or lyophilisate of nanoparticles as obtained by a process as claimed in any one of embodiments 1 to 18.

Embodiment 20: A nanoparticle of formula (II) below [Chem. 2]:

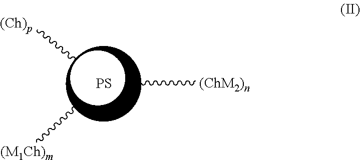

wherein:
   PS is an organic or inorganic polymer matrix, for example a polysiloxane matrix,
   [Ch-M₁] is a chelating group Ch complexed with a metal cation M₁ with a high atomic number Z greater than 40, and preferably greater than 50, for example a gadolinium cation,
[Ch-M₂] is a chelating group Ch complexed with a cation M₂ different from the metal cation M₁, for example chosen from metal cations with a high atomic number Z greater than 40, and preferably greater than 50, or chosen from radioactive isotopes, preferably M₂ is a bismuth cation,
   [Ch] is an uncomplexed Ch chelating group
characterized in that
(i) the chelating agents Ch are covalently grafted to the surface of the polymer matrix,
(ii) the molar ratio n/(n+m+p) is between 10% and 90%, preferably between 25% and 35%, the molar ratio m/(n+m+p) is between 10% and 90%, preferably between 65% and 75%, and the molar ratio p/(n+m+p) is substantially zero, and,
(iii) the average hydrodynamic diameter of the nanoparticle is between 1 and 50 nm, preferably between 2 and 20 nm, and more preferentially between 2 and 8 nm.
Embodiment 21: The nanoparticle as claimed in embodiment 20, characterized in that the metal cation M₁, and where appropriate M₂, is chosen from radiosensitizers and/or contrast agents for magnetic resonance imaging, in particular gadolinium or bismuth.

Embodiment 22: The nanoparticle as claimed in either one of embodiments 20 and 21, characterized in that the chelating group Ch is chosen from macrocyclic agents, preferably from 1,4,7-triazacyclononane-triacetic acid (NOTA), 1,4,7,10-tetraazacyclododecane-1,4,7,10-tetraacetic acid (DOTA), 1,4,7-triazacyclononane-1-glutaric acid-4,7-diacetic acid (NODAGA), and 1,4,7,10-tetraazacyclododecane, 1-(glutaric acid)-4,7,10-triacetic acid (DOTAGA), 2,2′,2″,2′″-(1,4,7,10-tetraazacyclododecane-1,4,7,10-tetrayl)tetraacetamide (DOTAM), and 1,4,8,11-tetraazacyclotetradecane (Cyclam), 1,4,7,10-tetraazacyclododecane (Cyclen) and deferoxamine (DFO).

Embodiment 23: The nanoparticle as claimed in any one of embodiments 20 to 22, characterized in that the chelating group Ch is DOTAGA of formula (I) below [Chem. 1]

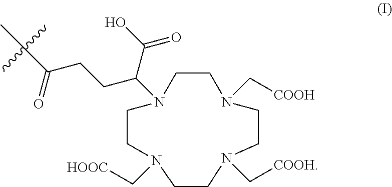 [SMILES: *C(=O)CCC(C(=O)O)N1CCN(CC(=O)O)CCN(CC(=O)O)CCN(CC(=O)O)CC1]

Embodiment 24: The nanoparticle as claimed in one of embodiments 20 to 23, characterized in that PS is a polysiloxane matrix.

Embodiment 25: The nanoparticle as claimed in embodiment 24, characterized in that
the weight ratio of silicon to the total weight of the nanoparticle is between 5% and 25%,
the total number n+m+p of chelating groups grafted onto the polymer is between 5 and 50 per nanoparticle, preferably between 10 and 30, and,
an average diameter of between 2 and 8 nm.

Embodiment 26: The nanoparticle as claimed in any one of embodiments 20 to 25, characterized in that the metal cation M₂ is chosen from scintigraphy imaging agents, for example ⁴⁴Sc, ⁶⁴Cu, ⁶⁸Ga, ⁸⁹Zr, ¹¹¹In, ^{99m}Tc.

Embodiment 27: The nanoparticle as claimed in any one of embodiments 20 to 25, characterized in that the metal cation M₂ is chosen from therapeutic agents for brachytherapy, for example ⁹⁰Y, ¹⁶⁶Ho, ¹⁷⁷Lu, ²¹²Bi, ²¹³Bi, ²¹²At.

Embodiment 28: The nanoparticle as claimed in any one of embodiments 20 to 27, characterized in that (i) PS is a polysiloxane matrix,
(ii) Ch is a DOTAGA chelating group of formula (I) below [Chem. 1]

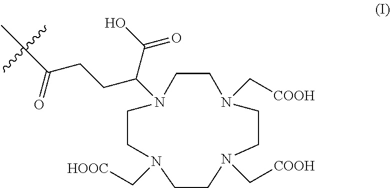 [SMILES: *C(=O)CCC(C(=O)O)N1CCN(CC(=O)O)CCN(CC(=O)O)CCN(CC(=O)O)CC1]

and grafted to the polysiloxane matrix by Si—C bond,
(iii) M₁ is the gadolinium cation Gd³⁺,
(iv) p is zero, and m+n is between 5 and 50, preferably between 10 and 30,
(v) n/n+p is between 0.1 and 0.9, for example between 0.25 and 0.35 or between 0.65 and 0.75, or between 0.45 and 0.55, and
(vi) the average hydrodynamic diameter is between 2 and 8 nm.

Embodiment 29: The nanoparticle as claimed in any one of embodiments 20 to 28, characterized in that (i) PS is a polysiloxane matrix,
(ii) Ch1 is a DOTAGA chelating group of formula (I) below [Chem. 1]

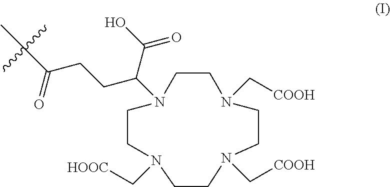 [SMILES: *C(=O)CCC(C(=O)O)N1CCN(CC(=O)O)CCN(CC(=O)O)CCN(CC(=O)O)CC1]

and grafted to the polysiloxane matrix by Si—C bond,
(iii) M₁ is the gadolinium cation Gd³⁺, M₂ is the bismuth cation Bi³⁺,
(iv) n+m+p is between 5 and 50, preferably between 10 and 30,
(v) n/(n+m+p) is between 10% and 90%, preferably between 45% and 55%,
(vi) m/(n+m+p) is between 10% and 90%, preferably between 45% and 55%,
(vii) p is substantially zero, and
(viii) the average hydrodynamic diameter is between 2 and 8 nm.

Embodiment 30: A colloidal solution of nanoparticles as claimed in any one of embodiments 20 to 29.

Embodiment 31: A pharmaceutical composition comprising a colloidal solution of nanoparticles as claimed in any one of embodiments 20 to 29, and one or more pharmaceutically acceptable excipients.

Embodiment 32: A pharmaceutical composition as claimed in embodiment 31, for the use thereof in the detection and or treatment of cancer in a subject, characterized in that said composition comprises an effective amount of metal cation M₁ and, where appropriate, of cations M₂, as radiosensitizer, preferably M₁ being gadolinium,
and in that the subject is treated with radiotherapy after administration of said composition.

### BRIEF DESCRIPTION OF THE DRAWINGS

Other features, details and advantages will become apparent on reading the detailed description below, and on analyzing the appended drawings, in which:

FIG. 1 shows the results of the intermediate titration of free DOTAs throughout the whole Gd/Bi nanoparticle formation process: 100/0 (A.) 70/30 (B.), 50/50 (C.), 30/70 (D.).

### DESCRIPTION OF THE EMBODIMENTS

Ultrafine Nanoparticles and AGuIX Nanoparticles

In a more particularly preferred embodiment, due in particular to their very small size and their stability and their biodistribution, the precursor nanoparticles that can be used in the processes of the present disclosure are nanoparticles comprising a polysiloxane matrix PS and which do not comprise a core based on metal oxide, unlike the core-shell nanoparticles comprising a core based on metal oxide and a polysiloxane coating (which are described in particular in WO2005/088314 and WO2009/053644).

Also, in a specific embodiment, the precursor nanoparticles that can be used according to the process of the present disclosure are polysiloxane-based gadolinium-chelated nanoparticles, of formula [Ch-M₁]ₙ-PS, wherein
(i) PS is a polysiloxane matrix,
(ii) Ch is a DOTAGA chelating group of formula (I) below [Chem. 1]

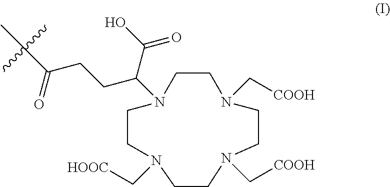 [SMILES: *C(=O)CCC(C(=O)O)N1CCN(CC(=O)O)CCN(CC(=O)O)CCN(CC(=O)O)CC1]

and grafted to the polysiloxane matrix by covalent bond,
(iii) M₁ is the gadolinium cation Gd³⁺,
(iv) n is between 5 and 50, preferably between 10 and 30, and
(v) the average hydrodynamic diameter is between 2 and 8 nm.

More specifically, these polysiloxane-based gadolinium-chelated nanoparticles are ultrafine nanoparticles obtained from AGuIX nanoparticles as starting material.

Such ultrafine AGuIX nanoparticles can be obtained by a top-down synthesis method described in particular in Mignot et al., Chem. Eur. J. 2013 “A Top-Down Synthesis Route to Ultrasmall Multifunctional Gd-Based Silica Nanoparticles for Theranostic Applications” DOI: 10.1002/chem.201203003.

Other ultrafine nanoparticle synthesis methods are also described in WO2011/135101, WO2018/224684 and WO2019/008040.

The AGuIX nanoparticles, which can be used as starting material in the process according to the present disclosure, have in particular formula (III) below [Chem. 3]

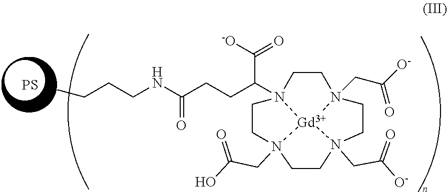

wherein PS is a polysiloxane matrix, and n is on average, around 10±2, and the nanoparticles have an average hydrodynamic diameter of 4±2 nm and a mass of around 10 kDa.

The AGuIX nanoparticles can also be characterized by the formula (IV) below [Chem. 4]

(GdSi₄₋₇C₂₄₋₃₀N₅₋₈O₁₅₋₂₅H₄₀₋₆₀, 5-10 H₂O)ₓ (IV)

In a preferred embodiment, the precursor nanoparticles are ultrafine or AGuIX nanoparticles as defined in the previous section and are complexed with the gadolinium cation.

The nanoparticles obtained according to the above process can then be advantageously functionalized with other chelating groups, different from Ch and/or targeting agents or hydrophilic molecules. It is therefore one of the aspects of the process of the present disclosure to provide a method for obtaining nanoparticles with advantageous properties, in particular for the use thereof as a drug or diagnostic or theragnostic agent, as described below.

Variant of the Process for the Production of Nanoparticles Comprising Metal Cations M₁ and M₂ Complexed with the Chelating Groups Ch

In one embodiment, the nanoparticles obtained according to the above process are brought into contact with a cation M₂, different from the metal cation M₁, for example a metal cation or a radioisotope of interest, in order to obtain the complexation of at least some of the chelating groups Ch freed following the treatment step (2).

Thus, the present disclosure relates to a process for preparing a colloidal solution of nanoparticles, each nanoparticle comprising chelating groups grafted onto a polymer matrix, a first fraction f1 of the chelating groups being complexed with a metal cation M₁, a second fraction f2 being being complexed with a cation M₂ and a third fraction f3 being uncomplexed, said process comprising
(1) the synthesis or the provision of a colloidal solution of precursor nanoparticles, said precursor nanoparticles having the following formula [Ch-M₁]ₙ-PS wherein:
   PS is an organic or inorganic polymer matrix,
   Ch is a chelating group complexed with a metal cation M1 with a high atomic number Z greater than 40, and preferably greater than 50,
   Ch is grafted onto the polymer matrix,
   n is between 5 and 100, and,
   the average hydrodynamic diameter of the precursor nanoparticle is between 1 and 50 nm, preferably between 2 and 20 nm, and more preferentially between 2 and 8 nm
(2) a step of treating the colloidal solution in an acid medium, for example by adding a hydrochloric acid solution, in order to obtain a pH preferably below 2.0, for a time sufficient to obtain a partial release of the metal cations M₁,
(3) where appropriate, a step of diluting the solution, for example with water,
(4) a purification step to separate the nanoparticles obtained in step (2) from the free metal cations M₁,
(5) where appropriate, a step of concentrating the solution of the nanoparticles obtained in step (4),
(6) where appropriate, repeating steps (3), (4) and (5),
(7) optionally, a step of partial recomplexation of the nanoparticles obtained in step (2), (3), (4), (5) or (6) with a determined amount of metal cation M₁ in order to obtain a determined amount of chelating group Ch complexed with the metal cation M₁,
(8) bringing the solution of nanoparticles obtained in step (2), (3), (4), (5) or (6) into contact with a sufficient amount of cation M₂, for example a metal cation different from the metal cations M₁ or a radioisotope of interest, to complex at least some of the chelating groups Ch1 freed in step (2), so as to obtain a colloidal solution of nanoparticles, each nanoparticle comprising chelating groups grafted onto a polymer matrix, a first fraction f1 of the chelating groups being complexed with a metal cation M₁, a second fraction f2 being being complexed with a cation M₂ and a third fraction f3 being uncomplexed and,
(9) where appropriate, freezing and/or freeze-drying the solution of nanoparticles obtained in step (8).

Specific embodiments of the process according to the present disclosure are given in the examples. Typically in the two embodiments of the process, whether there is a recomplexation with a metal M₂ (embodiment 8) or no recomplexation (embodiment 1), in step (2), those skilled in the art will be able to adapt the pH, and/or the treatment time depending on the desired amount of release of the metal cations M₁. It may also be possible to release a greater amount of metal cation M₁ in steps (2) to (6) and to adjust the desired fraction f1 with step (7) of partial recomplexation with the metal cation M₁. Indeed, depending in particular on the desired proportion of Ch group to be decomplexed, the duration of the acid treatment of step (2) will have to be adjusted by those skilled in the art. Typically, those skilled in the art will be able to monitor the decomplexation by an analysis method of their choice, for example by HPLC-ICP/MS. In other words, during step (2), the sufficient duration can be determined by monitoring said release by an analysis technique such as HPLC-ICP/MS.

In a specific embodiment, in particular with the use of AGuIX nanoparticles as precursor nanoparticles, the treatment time in step (2) is between 0.5 and 90 hours, for example between 1 and 72 hours, in particular at least 4, 5, 24 or 72 hours, at a pH below 2.0, preferably below 1.0.

Steps (7) and (8) may require restoring a pH between 6.0 and 8.0, preferably a neutral pH, and/or heating the solution of nanoparticles to a temperature and for a time sufficient to obtain the complexation. For example, step (7) or (8) can be carried out at a temperature of between 60° C. and 95° C., typically 80° C. for a time of between 24 and 72 hours, for example 48 hours.

Those skilled in the art will also be able to adapt the amount of cation M₂ in step (8) as a function of the amount of free chelating groups, and the desired fractions f2 and f3, respectively representing the fraction of chelating agent complexed with the metal cation M₂ or radioisotope and the amount of chelating agent remaining uncomplexed.

In one embodiment, those skilled in the art will use an excess amount of cations M₂ so as to complex essentially all the free chelating agents. Thus, the fraction f3 is substantially zero.

In another embodiment, if free chelating groups remain after step (8) of complexation with a cation M₂, it could also be possible to carry out another step of complexation with a metal cation M₁, in order to adapt the desired fractions f2 and f3.

Variant of the Process for Functionalizing the Nanoparticles with Targeting Molecules

In addition to the chelating functionalization, the nanoparticles obtained in the process according to the present disclosure can optionally be modified (functionalization) at the surface by hydrophilic compounds (PEG) and/or be charged differently to adapt their biodistribution within the body and/or targeting molecules to allow specific cell targeting, in particular for the targeting of specific tumour cells or tissues. The targeting agents are grafted to the polymer matrix and are present preferentially in a proportion of between 1 and 20 targeting agents per nanoparticle and preferably between 1 and 5 targeting agents.

For surface grafting of the targeting molecules, use may be made of conventional coupling with reactive groups present, optionally preceded by an activation step. The coupling reactions are known to those skilled in the art and will be chosen as a function of the structure of the surface layer of the nanoparticle and of the functional groups of the targeting molecule. See, for example, “Bioconjugate Techniques”, G. T Hermanson, Academic Press, 1996, in “Fluorescent and Luminescent Probes for Biological Activity”, Second Edition, W. T. Mason, ed. Academic Press, 1999. Preferred coupling methods are described below. Preferably, these targeting molecules are grafted to the amine bonds of the nanoparticles depending on the variant of the ultrafine or AGuIX nanoparticles as described in the preceding paragraph. The targeting molecules will be chosen as a function of the intended application.

In a specific embodiment, the precursor nanoparticles are functionalized with a targeting agent, such as a peptide, an immunoglobulin, a nanobody, an antibody, an aptamer or any other protein targeting, for example, tumor areas, typically an antibody, immunoglobulin or nanobody, VHH fragment, or “single domain”, targeting tumor-associated antigens or certain cancer markers known to those skilled in the art.

Nanoparticles Comprising Cations M₁ and M₂ Complexed with a Chelating Group Ch

The present disclosure also relates to nanoparticles and solutions of nanoparticles as obtained by the processes described in the preceding paragraphs, or capable of being obtained by the processes described in the preceding paragraphs.

Thus, the present disclosure relates to nanoparticles of formula (II) below [Chem. 2]

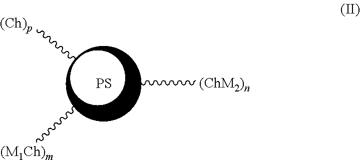

wherein:
   PS is an organic or inorganic polymer matrix, for example a polysiloxane matrix,
   [Ch-M₁] is a chelating group Ch complexed with a metal cation M₁ with a high atomic number Z greater than 40, and preferably greater than 50, for example a gadolinium cation,
   [Ch-M₂] is a chelating group Ch complexed with a cation M₂ identical to or different from the metal cation M₁, for example a metal cation with a high atomic number Z greater than 40, and preferably greater than 50, or a radioactive isotope, for example M₂ is a bismuth cation,
   [Ch] is an uncomplexed Ch chelating group and characterized in that
(i) the chelating agents Ch are covalently grafted to the surface of the polymer matrix,
(ii) the molar ratio n/(n+m+p) is between 10% and 90%, preferably between 25% and 35%, typically 30%, the molar ratio m/(n+m+p) is between 10% and 90%, preferably between 65% and 75%, and,
(iii) the average hydrodynamic diameter of the nanoparticle is between 1 and 50 nm, preferably between 2 and 20 nm, and more preferentially between 2 and 8 nm.

In one embodiment which can be preferentially combined with the preceding embodiment, the molar ratio p/(n+m+p) is substantially zero.

In another preferred embodiment which can be combined with the two preceding embodiments, the molar ratio m/(n+m+p) is between 45% and 55%, typically 50%, the molar ratio n/(n+m+p) is between 45% and 55%, typically 50%, and the molar ratio p/(n+m+p) is substantially zero.

The characteristics regarding in particular the chemical nature of the polymer matrix PS, the average hydrodynamic diameter, the chelating group Ch, and the number of chelating groups per nanoparticle, namely n+m+p, will be intrinsically associated with the choice of precursor nanoparticles in the process as described in the previous paragraph. They therefore also apply to nanoparticles as obtained by the process, or capable of being obtained by the process.

The nanoparticles preferably have very small diameters, for example between 1 and 10 nm, preferably between 2 and 8 nm.

The nanoparticles are also preferably nanoparticles comprising a polysiloxane matrix.

In a preferred embodiment, the chelating group Ch is DOTAGA of formula (I) below [Chem. 1]:

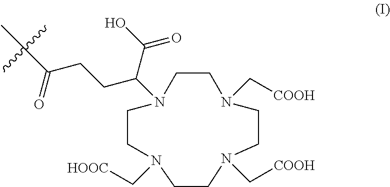 [SMILES: *C(=O)CCC(C(=O)O)N1CCN(CC(=O)O)CCN(CC(=O)O)CCN(CC(=O)O)CC1]

More particularly, the metal cations M₁ and M₂ are chosen independently from heavy metals, preferably from the group consisting of: Pt, Pd, Sn, Ta, Zr, Tb, Tm, Ce, Dy, Er, Eu, La, Nd, Pr, Lu, Yb, Bi, Hf, Ho, Sm, In and Gd. Preferably, the metal cations M₁ and M₂ (or M₂ and M₁) are Gd and Bi respectively.

In a particular embodiment, the nanoparticle comprises between 3 and 100, preferably between 5 and 50 metal cations M₁ and M₂, for example between 10 and 30, in particular of Gd and Bi.

In another embodiment, M₁ is chosen from the heavy metals as indicated above and M₂ is chosen from radioactive isotopes, in particular for the use thereof for scintigraphy imaging or brachytherapy.

Those skilled in the art will select the n/(n+m+p) and m/(n+m+p) molar ratios depending on the desired effect, and notably depending on the desired treatment, the type of patient, the dose used, and/or the patient to be treated. For example, in a particular embodiment, the (n+m)/(n+m+p) ratio is greater than or equal to 80%; notably between 90 and 100.

In a preferred embodiment, the nanoparticles of formula (2) above are characterized in that
(i) PS is a polysiloxane matrix,
(ii) Ch1 is a DOTAGA chelating group of formula (I) below [Chem. 1]

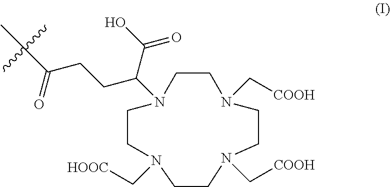 [SMILES: *C(=O)CCC(C(=O)O)N1CCN(CC(=O)O)CCN(CC(=O)O)CCN(CC(=O)O)CC1]

and grafted to the polysiloxane matrix by Si—C bond,
(iii) M₁ is the gadolinium cation Gd³⁺, M₂ is the bismuth cation Bi³⁺,
(iv) n+m+p is between 5 and 50, preferably between 10 and 30,
(v) n/(n+m+p) is between 10% and 90%, preferably between 45% and 55%,
(vi) m/(n+m+p) is between 10% and 90%, preferably between 45% and 55%,
(vii) p is substantially zero, and
(viii) the average hydrodynamic diameter is between 2 and 8 nm.

In a preferred embodiment, the nanoparticles of formula (2) above are characterized in that
(i) PS is a polysiloxane matrix,
(ii) Ch1 is a DOTAGA chelating group of formula (I) below [Chem. 1]

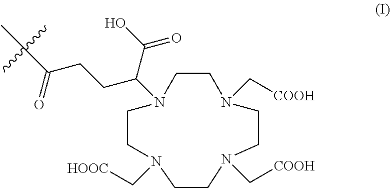 [SMILES: *C(=O)CCC(C(=O)O)N1CCN(CC(=O)O)CCN(CC(=O)O)CCN(CC(=O)O)CC1]

and grafted to the polysiloxane matrix by Si—C bond,
(iii) M₁ is the gadolinium cation Gd³⁺, M₂ is the bismuth cation Bi³⁺,
(iv) n+m+p is between 5 and 50, preferably between 10 and 30,
(v) n/(n+m+p) is between 25% and 35%,
(vi) m/(n+m+p) is between 65% and 75%,
(vii) p is substantially zero, and
(viii) the average hydrodynamic diameter is between 2 and 8 nm.

In a preferred embodiment, the nanoparticles of formula (2) above are characterized in that
(i) PS is a polysiloxane matrix,
(ii) Ch1 is a DOTAGA chelating group of formula (I) below [Chem. 1]

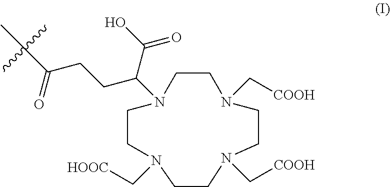 [SMILES: *C(=O)CCC(C(=O)O)N1CCN(CC(=O)O)CCN(CC(=O)O)CCN(CC(=O)O)CC1]

and grafted to the polysiloxane matrix by Si—C bond,
(iii) M₁ is the gadolinium cation Gd³⁺, M₂ is the bismuth cation Bi³⁺,
(iv) n+m+p is between 5 and 50, preferably between 10 and 30,
(v) n/(n+m+p) is between 65% and 75%,
(vi) m/(n+m+p) is between 25% and 35%,
(vii) p is substantially zero, and
(viii) the average hydrodynamic diameter is between 2 and 8 nm.

### Pharmaceutical Formulations of the Nanoparticles According to the Present Disclosure

The compositions comprising the nanoparticles according to the present disclosure are administered in the form of colloidal suspensions of nanoparticles. They can be prepared as described here or according to other methods known to those skilled in the art and administered via different routes, local or systemic, depending on the treatment and the area to be treated.

Also, the present disclosure relates to a colloidal suspension of nanoparticles of formula (2) as described in the preceding sections and the pharmaceutical compositions comprising these colloidal suspensions, where appropriate, in combination with one or more pharmaceutically acceptable excipients.

The pharmaceutical compositions can in particular be formulated in the form of lyophilized powders, or aqueous solutions for intravenous injection. In a preferred embodiment, the pharmaceutical composition comprises a colloidal solution with a therapeutically effective amount of nanoparticles of formula (2) as described in the preceding sections, in particular polysiloxane-based nanoparticles chelated with gadolinium and at least one other metal cation, for example bismuth, and more specifically, as obtained from AGuIX nanoparticles as described above.

In some embodiments, it is lyophilized powder, comprising between 200 mg and 15 g per vial, preferably between 250 and 1250 mg of nanoparticles. The powder may also comprise other excipients, and in particular CaCl₂.

Lyophilized powders can be reconstituted in an aqueous solution, typically sterile water for injection. Thus, the present disclosure relates to a pharmaceutical composition for use thereof as a solution for injection, comprising, as active principle, the nanoparticles of formula (2) as described in the preceding sections, in particular polysiloxane-based gadolinium-chelated nanoparticles, and more specifically, as obtained from AGuIX nanoparticles as described above.

### Uses of the Nanoparticles

Due to the presence of chelating groups Ch1 that are free or complexed with metal cations M₁, and, where appropriate, cations M₂, the nanoparticles according to the present disclosure allow use as a radiosensitizer when M₁ and/or M₂ are judiciously chosen for use as a radiosensitizer, and the process comprises, after administration of the composition, a step of irradiating the subject with an effective dose for the treatment of the tumor by radiotherapy.

In some embodiments, the nanoparticles according to the present disclosure allow use as an imaging agent, for medical imaging, for example, magnetic resonance imaging (MRI), in particular for the detection of tumors in a subject, when M₁ and/or M₂ are judiciously chosen for use as imaging agent, for example contrast agent for MRI, and the process comprises, after administration of the composition, a step of imaging the subject with an effective dose for imaging the area of interest, in particular MRI imaging in a subject for the detection of tumors.

A “patient” or “subject”, it is preferably understood to mean a mammal or a human being including for example a subject having a tumor.

The terms “treatment” and “therapy”, refer to any act which aims to improve the health of a patient, such as therapy, prevention, prophylaxis, and slowing down a disease. In some cases, these terms refer to the improvement or eradication of a disease or of the symptoms associated with the disease. In other embodiments, these terms refer to the reduction in the spread or exacerbation of the disease resulting from the administration of one or more therapeutic agents to a subject afflicted with such a disease. In the context of the treatment of tumors, the term “treatment” can typically include a treatment for stopping the growth of a tumor, the reducing the size of the tumor and/or for eliminating the tumor.

In particular, nanoparticles are used for the detection and/or treatment of solid tumours, for example brain cancer (primary and secondary, glioblastoma, etc.), liver cancers (primary and secondary), pelvic tumors (cervical cancer, prostate cancer, anorectal cancer, colorectal cancer), upper aerodigestive tract cancers, lung cancer, oesophageal cancer, breast cancer, pancreatic cancer.

An “effective amount” of nanoparticles refers to the amount of nanoparticles as described above which, when administered to a patient, is sufficient to be localized in the tumor and allow detection and/or treatment of the tumor by radiosensitizing effect with radiotherapy treatment.

This amount is determined and adjusted according to factors such as the age, sex and weight of the subject.

The administration of the nanoparticles as described above can be carried out by the intratumoral, subcutaneous, intramuscular, intravenous, intradermal, intraperitoneal, oral, sublingual, rectal, vaginal or intranasal route, by inhalation or by transdermal application. Preferably, it is performed intratumorally and/or intravenously.

The irradiation methods for the treatment of tumors after administration of nanoparticles as radiosensitizer are well known to those skilled in the art and have been described in particular in the following publications: WO2018/224684, WO2019/008040 et C. Verry, et al., Science Advances, 2020, 6, eaay5279; and, C. Verry, et al, NANO-RAD, a phase I study protocol, BMJ Open, 2019, 9, e023591.

The total dose of irradiation during radiotherapy will be adjusted depending on the type of cancer, the stage and the subject to be treated. For a curative dose, a typical total dose for a solid tumor is of the order of 20 to 120 Gy. Other factors may be taken into account such as chemotherapy treatment, co-morbidity, and/or whether radiotherapy takes place before or after surgery. The total dose is usually divided. The radiotherapy step in the process according to the present disclosure may comprise, for example, several fractions between 2 and 6 Gy per day, for example 5 days per week, and in particular over 2 to 8 consecutive weeks, the total dose possibly being between 20 and 40 Gy, for example 30 Gy.

Also, the present disclosure relates to a method for treating tumors, in particular solid tumors, in a subject who has need thereof, said method comprising the administration to the subject of an effective amount of nanoparticles of formula (2) as described above above, and for which M₁ and M₂ are chosen from magnetic resonance imaging agents and radiosensitizers, in particular gadolinium and bismuth.

The nanoparticles according to the present disclosure can be administered alone, or in combination with one or more other active principles, and in particular other drugs such as cytotoxic or antiproliferative agents or other anticancer agents and in particular immune checkpoint inhibitors. Combined administration is understood to mean simultaneous administration or sequential administration (at different times).

### EXAMPLES

### Materials and Methods

Acidix products are obtained by introducing the starting product AGuIX®, supplied by Nh Theraguix (France), into a strongly acidic medium obtained from extra-pure 37% hydrochloric acid from CarlRoth.

The filtration steps are carried out using a peristaltic pump and a Vivaflow 200®—5 kDa cassette from Sartorius Stedim Biotech (France) used as under the conditions described in the instructions linked to the Vivaflow 200® product.

The measurement of the hydrodynamic diameter and also the titration of the isoelectric point are carried out with a Zetasizer Nano-S (633 nmHe-Ne laser) from Malvern Instruments (USA). For the measurement of the isoelectric point, this apparatus is coupled to an MPT-2 automatic titrator from Malvern Instruments (USA).

The HPLC-UV is carried out with an Agilent 1200 with a DAD detector. The reverse phase column used is a C4, 5 μm, 300 Å, 150×4.6 mm column from Jupiter. Detection is performed by a UV detector at a wavelength of 295 nm. The gradient of the A (H₂O/ACN/TFA: 98.9/1/0.1) and B (H₂O/ACN/TFA: 10/89.9/0.1) phases is as follows: 5 minutes at 95/5 followed by a linear gradient over 10 min which makes it possible to reach the 10/90 ratio which is maintained for 15 minutes. At the end of these 15 minutes, the ratio of A is returned to 95% in 1 minute and is followed by a 7-minute plateau at 95/5. The products used in the composition of the eluting phases are all HPLC grade certified.

The elemental analysis was carried out at the Institut des Sciences Analytiques [Institute of Analytical Sciences], UMR 5280, Pole Isotopes & Organique, 5 rue de la Doua 69100 Villeurbanne.

The HPLC-ICP/MS is performed with Nexion 2000 from Perkin-Elmer (USA). The measurement of the free elements in the medium is carried out in isocratic mode with an elution phase of the following composition: 95% A and 5% B. The composition of phases A and B is identical to the HPLC-UV method. The reverse phase column used is a C4, 5 μm, 300 Å, 150×4.6 mm column from Jupiter. The products used in the composition of the eluting phases are all HPLC grade certified.

The freeze-drying of the particles is carried out using an Alpha 2-4 LSC freeze-dryer from Christ (Germany) following the “main drying” program.

The free DOTA is measured by adding an increasing amount of Cu²⁺ to a fixed amount of product. The copper comes from a 15 mM solution of Cu²⁺ previously prepared from CuCl₂ (Sigma Aldrich, 99%, powder, 25 g) dissolved in ultrapure water. The volume of the samples is then adjusted with an acetate buffer solution at pH 5 to ensure complete complexation. Once the samples have been prepared, an HPLC-UV measurement is carried out at 295 nm as specified previously. The total absorbance is measured by integrating the 0-15 min segment of the chromatogram obtained. Since the increase in the absorbance signal is based on the formation of the DOTA(Cu) complex, the amount of free DOTA in the medium can be obtained when the stoichiometric point between free DOTA and added Cu²⁺ is reached. This point results in an abrupt change in slope on the graph obtained.

### Example 1: Acidification of the Medium and Release of Gd³⁺ Ion

In order to obtain a nanoparticle according to the process, the AGuIX® product was placed in an acid medium in order to protonate the DOTA groups and thus release some of the Gd³⁺ ions initially complexed.

First, a 200 g/L solution of AGuIX® was prepared by dissolving 10 g of product in 50 ml of ultrapure water. The solution was left stirring at room temperature for 1 hour. At the same time, a 2M hydrochloric acid solution was prepared by adding 10 ml of 37% hydrochloric acid (37% Hydrochloric acid, extra pure, 2.5 L, plastic, CarlRoth) to 50 ml of ultrapure water.

After stirring for one hour, 50 ml of the 2M hydrochloric acid solution are added to the 50 ml of AGuIX®. The pH was then measured and is less than 0.5. The solution obtained is brown-orange in color. The combined mixture is left in an oven preheated to 50° C. for 4 hours. A sample was taken every hour to observe the release of gadolinium ions by HPLC-ICP/MS. It is observed that the peak of free Gd³⁺ in the medium at the retention time Tr=2.3 min increases with the reaction time.

### Example 2: Obtaining a Gadolinium-Free Nanoparticle

A 100 g/L solution of AGuIX® is prepared by dissolving 5 g of product in 50 ml of ultrapure water. The solution is left stirring at room temperature for 1 hour. At the same time, a 2M hydrochloric acid solution is prepared by adding 10 ml of 37% hydrochloric acid (37% Hydrochloric acid, extra pure, 2.5 L, plastic, CarlRoth) to 50 ml of ultrapure water.

After stirring for one hour, 50 ml of the 2M hydrochloric acid solution are added to the 50 ml of the solution containing AGuIX®. The combined mixture is heated at 50° C. for 1 hour. The 100 ml of solution thus obtained are purified using a peristaltic pump and a Sartorius Vivaflow 50 R-5 kDa cassette in order to separate the particles from the Gd³⁺ ions released and thus push the equilibrium toward the release of the Gd³⁺ still complexed. The volume of the initial solution is thus concentrated to 50 ml. The filtrate is directly analyzed by ICP-MS in order to estimate the amount of Gd³⁺ still present in the medium. The AGuIX solution is again rediluted with 50 ml of a 1M hydrochloric acid solution. Similarly, the solution is left at 50° C. for 1 hour then reconcentrated to 50 ml. The process is repeated until the amount of Gd³⁺ measured is zero. Once this level has been reached, the solution is purified by a factor of 10 000 using ultrapure water in order to eliminate excess salt due to the use of concentrated hydrochloric acid. At the end of the process, an ICP-MS measurement on the final product makes it possible to verify that it is free of any Gd³⁺.

Once the final product has been recovered, it is bottled, frozen at −80° C. and then freeze-dried. The powder obtained is then redispersed in ultrapure water to obtain a 100 g/L solution. A measurement of free DOTA is carried out by copper complexation and measurement of the absorbance at 295 nm. This measurement indicates that the new product has a free DOTA content of 71 μmol/mg of product. By way of comparison, the batch of AGuIX® used for this experiment contained 12.7% (wt %) of Gd³⁺, i.e. a DOTA(Gd) content of 81 μmol/mg of AGuIX.

Furthermore, a sample of the final product is sent to a specialized laboratory to check the content of Gd still present in the sample, the result indicates a weight content of Gd of 0.19% (wt %) (table 1) compared with 12.7% (wt %) for the abovementioned initial batch. The size of the nanoparticle obtained is measured by DLS and has an average hydrodynamic diameter of 5.2 nm±2.6 nm, of the same order as the original AGuIX® product. Furthermore, the isoelectric point of the final product is measured, the final product therefore has a neutral charge for a pH of 5.2. This pH is lower than the isoelectric point of AGuIX®, which is around 7. This decrease is in agreement with a generation of free DOTA on the surface.

TABLE 1
Elemental analysis in Gd in AGulX ® and the final product.

| | % Gd (wt %) |
|---|---|
| AGulX ® | 12.7 |
| Final | 0.19 |
| product | |

### Example 3: Release of 80% of the Complexed Gd: 72 h

The process presented in the previous example was adapted to have only a partial and controlled release of gadolinium. First, a 200 g/L solution of AGuIX® was prepared by dissolving 10 g of product in 50 ml of ultrapure water. The solution is left stirring at room temperature for 1 hour. At the same time, a 2M hydrochloric acid solution is prepared by adding 10 ml of 37% hydrochloric acid (37% Hydrochloric acid, extra pure, 2.5 L, plastic, CarlRoth) to 50 ml of ultrapure water.

After stirring for one hour, 50 ml of the 2M hydrochloric acid solution are added to the 50 ml of AGuIX®. The pH is then measured and is less than 0.5. The combined mixture is left in an oven preheated to 50° C. for 72 hours. A sample is taken after 4 h of reaction and also at the end of the reaction in order to observe the final release of the Gd³⁺ ions by HPLC-ICP/MS. The peak of free Gd³⁺ in the medium at the retention time Tr=2.3 min increases with the reaction time. The peak at 72 h covers 77.2% of the 12 ppm Gd³⁺ reference peak which corresponds to the total Gd concentration of our AGuIX solution used. Thus, 22.7% of the DOTAGA(Gd) complexes initially present remain in our particles.

### Example 4: Formation of Gd/Bi Nanoparticles and Complexation Monitoring

To produce a nanoparticle with a specific Gd/Bi ratio, the amount of free DOTA is measured. Thus, the starting product for particle formation is a nanoparticle where 80% of the DOTAs are free and the remaining 20% are complexed with Gd³⁺.

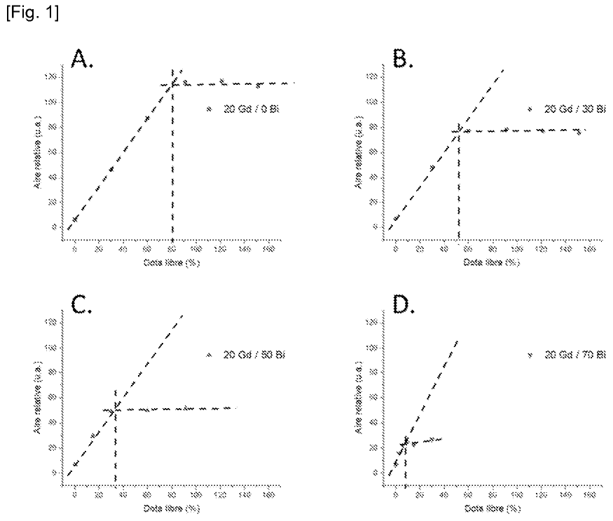

To produce the following three 30/70, 50/50 and 70/30 Gd/Bi (nGd/nBi) batches, the Bi complexation is carried out first. The Bi³⁺ complexation is a long process. Thus, of adding the required amount of BiCl₃ (Sigma-Aldrich, reagent grade, >98%) to reach the desired ratio, the pH is adjusted to 7 with a 1M NaOH solution and the mixture is placed at 80° C. for 48 hours. After each Bi³⁺ complexation step, the number of free DOTA remaining is measured by copper complexation in order to confirm the progress of the complexation (FIG. 1). Once the content of free DOTA remaining complies with the ratios, the required amount of GdCl₃.6H2O (Merck, 99%) is added to complex the rest of the DOTAs and thus form the desired particles. The addition of GdCl₃ is followed by readjusting the pH to 7 and by 24 hours at 80° C. The particles are then freeze-dried. Once the products have been obtained, a sample of each batch is sent to our partner for elemental analysis in order to verify the actual content of each element in the various batches (table 2)

TABLE 2
Results of the elemental analyses of the final particles

| Batch | % Gd (wt %)/% Bi (wt %) | nGd/nBi |
|---|---|---|
| Gd/Bi: 70/30 | 7.54%/4.24% | 70.6/29.4 |
| Gd/Bi: 50/50 | 5.38%/6.84% | 50.7/49.3 |
| Gd/Bi: 30/70 | 3.16%/9.97% | 29.9/70.1 |

The present technical solutions can be applied in particular in the field of medicine, in particular for the treatment of tumors.

The present disclosure is not limited to the examples described above, merely by way of example, but encompasses any variant that those skilled in the art are able to envision, within the scope of the claimed protection.

1. A process for preparing a colloidal solution of nanoparticles, each nanoparticle comprising chelating groups grafted onto a polymer matrix, one portion only of the chelating groups being complexed with a metal cation, the other portion being uncomplexed, said process comprising
(1) the synthesis or the provision of a colloidal solution of precursor nanoparticles, said precursor nanoparticles having the following formula [Ch-M₁]ₙ-PS wherein:
PS is an organic or inorganic polymer matrix, for example a polysiloxane matrix,
[Ch-M₁] is a chelating group complexed with a metal cation M₁ with a high atomic number Z greater than 40, and preferably greater than 50,
Ch is covalently grafted to the surface of the polymer matrix, for example, a polysiloxane matrix,
n is between 5 and 100, and,
the average hydrodynamic diameter of the nanoparticles is between 1 and 50 nm, preferably between 2 and 20 nm, and more preferentially between 2 and 8 nm,
(2) a step of treating the colloidal solution in an acid medium, for example by adding a hydrochloric acid solution, in order to obtain a pH preferably below 2.0, preferably below 1.0, for a time sufficient to obtain a partial release of the metal cations M₁,
(3) where appropriate, a step of diluting the colloidal solution, for example with water,
(4) a purification step to separate the nanoparticles obtained in step (2) from the metal cations M₁ released,
(5) where appropriate, a step of concentrating the solution of the nanoparticles obtained in step (4),
(6) where appropriate, repeating steps (3), (4) and (5),
(7) where appropriate, freezing and/or freeze-drying the solution of nanoparticles obtained in one of steps (4), (5) or (6).

2. The process as claimed in claim 1, wherein M₁ is chosen from metal cations selected from radiosensitizers and/or contrast agents for magnetic resonance imaging (MRI), for example M₁ is chosen from gadolinium and bismuth.

3. The process as claimed in claim 1, wherein the chelating group Ch is chosen from macrocyclic agents, preferably from 1,4,7-triazacyclononane-triacetic acid (NOTA), 1,4,7,10-tetraazacyclododecane-1,4,7,10-tetraacetic acid (DOTA), 1,4,7-triazacyclononane-1-glutaric acid-4,7-diacetic acid (NODAGA), and 1,4,7,10-tetraazacyclododecane, 1-(glutaric acid)-4,7,10-triacetic acid (DOTAGA), 2,2′,2″,2′″-(1,4,7,10-tetraazacyclododecane-1,4,7,10-tetrayl)tetraacetamide (DOTAM), and 1,4,8,11-tetraazacyclotetradecane (Cyclam), 1,4,7,10-tetraazacyclododecane (Cyclen) and deferoxamine (DFO).

4. The process as claimed in claim 1, wherein the chelating group Ch is DOTAGA of formula (I) below:

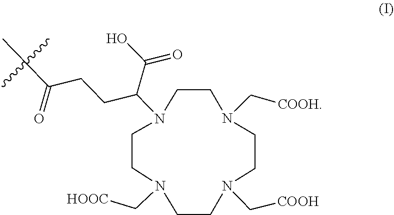 [SMILES: *C(=O)CCC(C(=O)O)N1CCN(CC(=O)O)CCN(CC(=O)O)CCN(CC(=O)O)CC1]

5. The process as claimed in claim 1, wherein PS is a polysiloxane matrix.

6. The process as claimed in claim 5, wherein the precursor nanoparticles have the following characteristics:
the weight ratio of silicon to the total weight of the nanoparticle is between 5% and 25%,
the total number n of chelating groups grafted to the polymer is between 5 and 50 per nanoparticle, preferably between 10 and 30, and,
the nanoparticle has an average diameter of between 2 and 8 nm.

7. The process as claimed in claim 1, wherein the precursor nanoparticles have the following characteristics:
(i) PS is a polysiloxane matrix,
(ii) Ch is a DOTAGA chelating group of the following formula [Chem. 1] 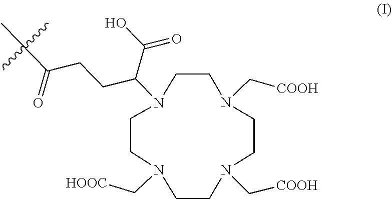 [SMILES: *C(=O)CCC(C(=O)O)N1CCN(CC(=O)O)CCN(CC(=O)O)CCN(CC(=O)O)CC1]
and grafted to the polysiloxane matrix by Si—C bond,
(iii) M₁ is the gadolinium cation Gd³,
(iv) n is between 5 and 50, preferably between 10 and 30, and
(v) the average hydrodynamic diameter is between 2 and 8 nm.

8. A process for preparing a colloidal solution of nanoparticles, each nanoparticle comprising chelating groups grafted onto a polymer matrix, a first fraction f1 of the chelating groups being complexed with a metal cation M₁, a second fraction f2 being being-complexed with a cation M₂, and a third fraction f3 being uncomplexed, said process comprising
(1) the synthesis or the provision of a colloidal solution of precursor nanoparticles, said precursor nanoparticles having the following formula [Ch-M₁]ₙ-PS wherein:
PS is an organic or inorganic polymer matrix,
Ch is a chelating group complexed with a metal cation M₁ with a high atomic number Z greater than 40, and preferably greater than 50,
Ch is grafted onto the polymer matrix,
n is between 5 and 100, and,
the average hydrodynamic diameter of the nanoparticle is between 1 and 50 nm, preferably between 2 and 20 nm, and more preferentially between 2 and 8 nm
(2) a step of treating the colloidal solution in an acid medium, for example by adding a hydrochloric acid solution, in order to obtain a pH below 2.0, preferably below 1.0, for a time sufficient to obtain a partial release of the metal cations M₁,
(3) where appropriate, a step of diluting the solution, for example with water,
(4) a purification step to separate the nanoparticles obtained in step (2) from the free metal cations M₁,
(5) where appropriate, a step of concentrating the solution of the nanoparticles obtained in step (4),
(6) where appropriate, repeating steps (3), (4) and (5),
(7) optionally, a step of partial recomplexation of the nanoparticles obtained in step (2), (3), (4), (5) or (6) with a determined amount of metal cation M₁ in order to obtain a determined amount of chelating group Ch complexed with the metal cation M₁,
(8) bringing the solution of nanoparticles obtained in step (4), (5), (6) or (7) into contact with a sufficient amount of cation M₂, preferably with a high atomic number Z greater than 40, and preferably greater than 50, for example a metal cation different from the metal cations M₁ or a radioisotope, to complex at least some of the chelating groups Ch1 freed in step (2) and,
(9) where appropriate, freezing and/or freeze-drying the solution of nanoparticles obtained in step (8).

9. The process as claimed in claim 8, wherein M₁ and/or M₂ are chosen from metal cations selected from radiosensitizers and/or contrast agents for magnetic resonance imaging (MRI), for example gadolinium or bismuth.

10. The process as claimed in claim 8, wherein the chelating group Ch is chosen from macrocyclic agents, preferably from 1,4,7-triazacyclononane-triacetic acid (NOTA), 1,4,7,10-tetraazacyclododecane-1,4,7,10-tetraacetic acid (DOTA), 1,4,7-triazacyclononane-1-glutaric acid-4,7-diacetic acid (NODAGA), and 1,4,7,10-tetraazacyclododecane, 1-(glutaric acid)-4,7,10-triacetic acid (DOTAGA), 2,2′,2″,2′″-(1,4,7,10-tetraazacyclododecane-1,4,7,10-tetrayl)tetraacetamide (DOTAM), and 1,4,8,11-tetraazacyclotetradecane (Cyclam), 1,4,7,10-tetraazacyclododecane (Cyclen) and deferoxamine (DFO).
